(19) United States
(12) Patent Application Publication
(10) Pub. No.: US 2008/0208155 A1
(43) Pub. Date: Aug. 28, 2008

(54) Diaper having abdominal stretch panels

(76) Inventors: Gary Dean LaVon, Liberty Township, OH (US); Thomas Henrich, Montgomery, OH (US); Kevin Michael Smith, Cincinnati, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/709,500

(22) Filed: Feb. 22, 2007

(51) Int. Cl. A61F 13/15

(52) U.S. Cl. 604/385.3

(57) ABSTRACT

A disposable absorbent article including a chassis and an absorbent assembly in combination with abdominal stretch panels extending circumferentially around the waist of the wearer. The chassis includes a water-impermeable backsheet that may be folded laterally inward at both of its side edges to form opposing side flaps. Each side flap is attached to the interior surface of the chassis adjacent to its end edges. Each side flap has a longitudinally extending elastic gathering member attached adjacent to its proximal edge. The chassis may include an extensible formed web material. The absorbent assembly may be attached in a cruciform pattern to the chassis to allow unattached portions of the chassis to extend laterally. Frangible tear lines and/or tear locator lines may be formed to facilitate removal of the article from the wearer.

### FIELD OF THE INVENTION

This invention relates to disposable absorbent articles such as disposable diapers and other articles intended for use on incontinent persons.

### BACKGROUND OF THE INVENTION

Disposable absorbent articles are designed to absorb and contain bodily waste in order to prevent soiling of the body and clothing of the wearer, as well as bedding or other objects with which the wearer comes into contact. As the usage of disposable absorbent articles has expanded, their complexity has increased with the incorporation of additional features serving to enhance their performance and appearance. The costs of the materials and the costs of the manufacturing processes have also increased in conjunction with the increase in complexity. As a result, the prices at which these articles are sold have risen to levels that many potential purchasers around the world cannot afford to pay. Thus, a need exists for a simple disposable absorbent article.

### SUMMARY OF THE INVENTION

A disposable absorbent article includes a chassis and an absorbent assembly in combination with abdominal stretch panels extending circumferentially around the waist of the wearer. The chassis includes a water-impermeable backsheet that may be folded laterally inward at both of its side edges to form opposing side flaps. Each side flap is attached to the interior surface of the chassis adjacent to its end edges. Each side flap has a longitudinally extending elastic gathering member attached adjacent to its proximal edge. The chassis may include an extensible formed web material. The absorbent assembly may be attached in a cruciform pattern to the chassis to allow unattached portions of the chassis to extend laterally.

### BRIEF DESCRIPTION OF THE DRAWINGS

In the accompanying drawing figures, like reference numerals identify like elements, which may or may not be identical in the several exemplary embodiments that are depicted. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description.

In the drawing figures and in the written description, lowercase letters appended to reference numerals indicate generally symmetric elements, e.g., left and right symmetric elements may be respectively identified by the reference numerals 1a and 1b. A reference numeral without an appended lowercase letter identifies all of the elements to which that particular reference numeral applies, e.g., the same elements as a group may be designated 1.

The edges of various components are shown offset from each other for clarity; the depiction of parallel edges immediately adjacent to each other is intended to represent that these edges are disposed either collinearly or in close proximity to each other.

FIG. 1 is a plan view of an exemplary disposable absorbent article in the form of a diaper 20, which is shown in its flat, uncontracted state, i.e., without the contraction induced by elastic members. In FIG. 1, the interior of the diaper is shown facing the viewer.

FIG. 2 is a plan view of the diaper 20 of FIG. 1 with the exterior portion of the diaper 20 shown facing the viewer.

FIG. 3 is a section view of the diaper 20 of FIG. 1 taken at the section line 3-3.

FIG. 4 is a section view of the diaper 20 of FIG. 1 taken at the section line 4-4.

FIG. 5 is a section view of the diaper 20 of FIG. 1 taken at the section line 5-5.

FIG. 6 is a section view of the diaper 20 of FIG. 1 taken at the section line 6-6.

FIG. 7 is a simplified side elevation view of an exemplary diaper 20 being worn about a lower torso of a wearer.

FIG. 8 is a front elevation view of the diaper 20 of FIG. 7.

FIG. 9 is a back elevation view of the diaper 20 of FIG. 7.

FIG. 10 is an elevation view of a laminate stretch panel.

FIG. 11 is a view of the laminate stretch panel of FIG. 10 in a stretched condition.

FIG. 12 is a plan view of an exemplary absorbent assembly 200 with the interior portion of the absorbent assembly 200 shown facing the viewer.

FIG. 13 is a view of the absorbent assembly of FIG. 12 taken at the section line 13-13.

FIG. 14 is a view of the absorbent assembly of FIG. 12 taken at the section line 14-14.

FIG. 15 is a section view of an exemplary absorbent assembly 200.

FIG. 16 is a section view of an exemplary absorbent assembly 200.

### DETAILED DESCRIPTION OF THE INVENTION

In this description, the following terms have the following meanings:

The term “absorbent article” refers to a device that absorbs and contains liquid, and more specifically, refers to a device that is placed against or in proximity to the body of the wearer to absorb and contain the various exudates discharged from the body.

The term “diaper” refers to an absorbent article that is generally worn by infants and incontinent persons about the lower torso and that is specifically adapted to receive and contain urinary and fecal waste.

The term “disposable” refers to the nature of absorbent articles that generally are not intended to be laundered or otherwise restored or reused as an absorbent article, i.e., they are intended to be discarded after a single use and, preferably, to be recycled, composted or otherwise disposed of in an environmentally compatible manner.

The term “longitudinal” refers to a direction running from a waist edge to an opposing waist edge of the article and generally parallel to the maximum linear dimension of the article. Directions within 45° of the longitudinal direction are considered to be “longitudinal”.

The term “lateral” refers to a direction running from a side edge to an opposing side edge of the article and generally at a right angle to the longitudinal direction. Directions within 45° of the lateral direction are considered to be “lateral”.

The term “circumferential” refers to a direction encircling the waist of the wearer generally parallel to the lateral direction.

The term “disposed” refers to an element being attached and positioned in a particular place or position in a unitary structure with other elements.

The term “attach” refers to elements being connected or united by adhering, bonding, etc., by any method suitable for the elements being attached together and their constituent materials. Many suitable methods for attaching elements together are well-known, including adhesive bonding, pressure bonding, thermal bonding, etc. Such attachment methods may be used to attach elements together over a particular area either continuously or intermittently. Unless indicated otherwise, elements that are described as being attached to each other are attached directly together, with either nothing or only bonding material, e.g., an adhesive, between them. Unless indicated otherwise, elements that are described as being attached to each other are attached permanently together, i.e., attached in such a way that one or both of the elements and/or any bonding material that is present must be damaged in order to separate them. This permanent attachment excludes temporary attachment, such as fastening elements together by means of fasteners that may be unfastened.

The term “laminate” refers to elements being attached together in a layered arrangement.

The terms “water-permeable” and “water-impermeable” refer to the penetrability of materials in the context of the intended usage of disposable absorbent articles. Specifically, the term “water-permeable” refers to a layer or a layered structure having pores, openings, and/or interconnected void spaces that permit liquid water to pass through its thickness in the absence of a forcing pressure. Conversely, the term “water-impermeable” refers to a layer or a layered structure through the thickness of which liquid water cannot pass in the absence of a forcing pressure. A layer or a layered structure that is water-impermeable according to this definition may be permeable to water vapor, i.e., may be “water vapor-permeable”. Such a water vapor-permeable layer or layered structure is commonly known in the art as “breathable”. As is well known in the art, a common method for measuring the permeability to water of the materials typically used in absorbent articles is a hydrostatic pressure test, also called a hydrostatic head test or simply a “hydrohead” test. Suitable well known compendial methods for hydrohead testing are approved by INDA (formerly the International Nonwovens and Disposables Association, now The Association of the Nonwoven Fabrics Industry) and EDANA (European Disposables and Nonwovens Association).

The terms “proximal” and “distal” refer respectively to the location of an element near to or far from the center of a structure, e.g., the proximal edge of a longitudinally extending element is located nearer to the longitudinal axis than the distal edge of the same element is located relative to the same longitudinal axis.

The terms “interior” and “exterior” refer respectively to the location of an element that is intended to be placed against or toward the body of a wearer when an absorbent article is worn and the location of an element that is intended to be placed against or toward any clothing that is worn over the absorbent article. Synonyms for “interior” and “exterior” include, respectively, “inner” and “outer”, as well as “inside” and “outside”. Also, when the absorbent article is oriented such that its interior faces upward, e.g., when it is laid out in preparation for setting the wearer on top of it, synonyms include “upper” and “lower”, “above” and “below”, “over” and “under”, and “top” and “bottom”, respectively.

The term “nonwoven” refers to a sheet, web, or batt of directionally or randomly oriented fibers, made by bonding or entangling the fibers through mechanical, thermal, or chemical means. Nonwoven materials exclude paper and products which are woven, knitted, tufted, or felted by wet milling. The fibers are preferably but not necessarily man-made synthetics.

The term “abdominal stretch panel” refers to a structural component that resists elongation by providing a circumferential contractive force around the waist opening of a diaper when it is stretched in the circumferential direction.

### DESCRIPTION OF EXEMPLARY DIAPER EMBODIMENT

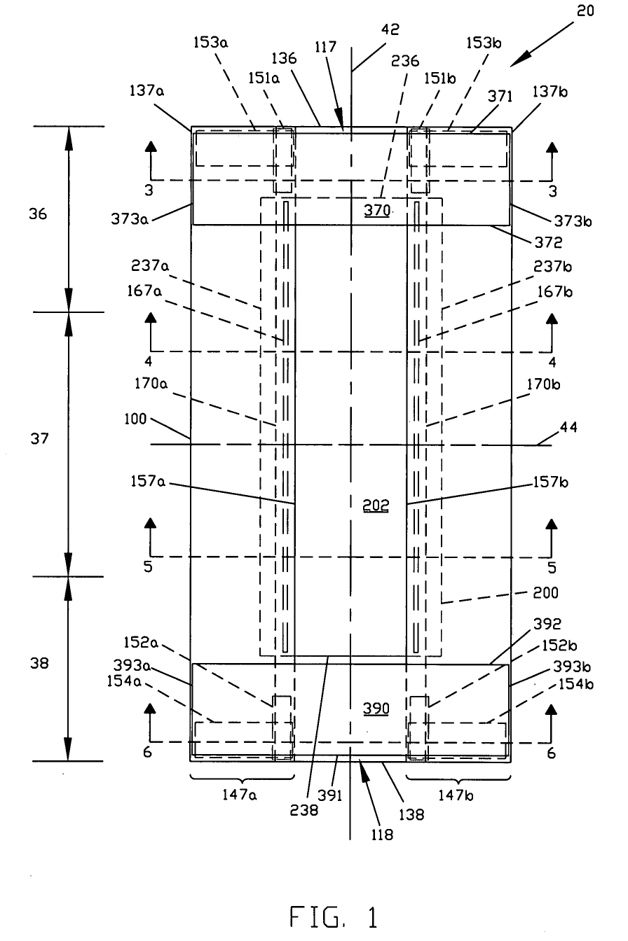

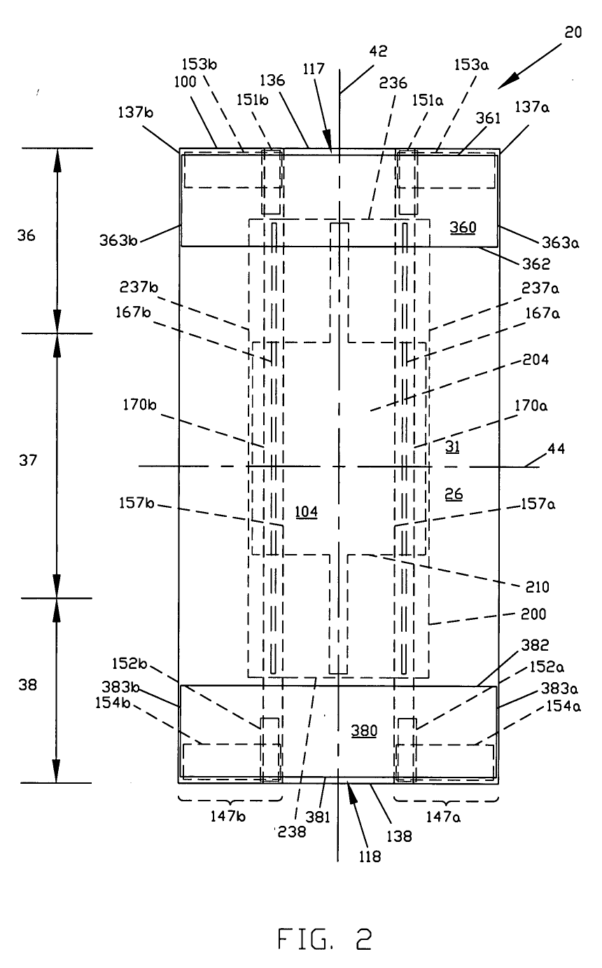

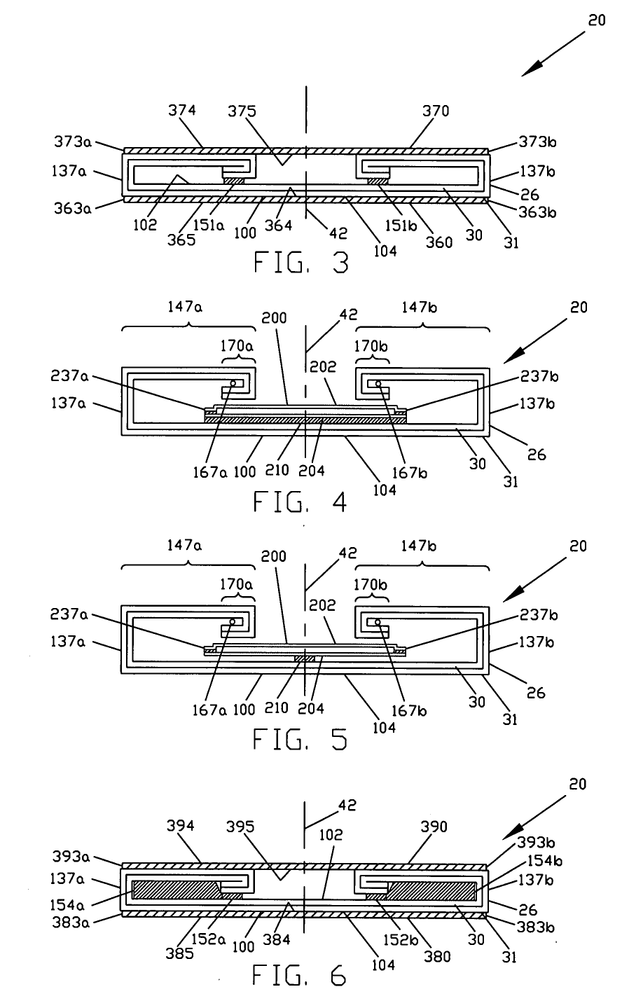

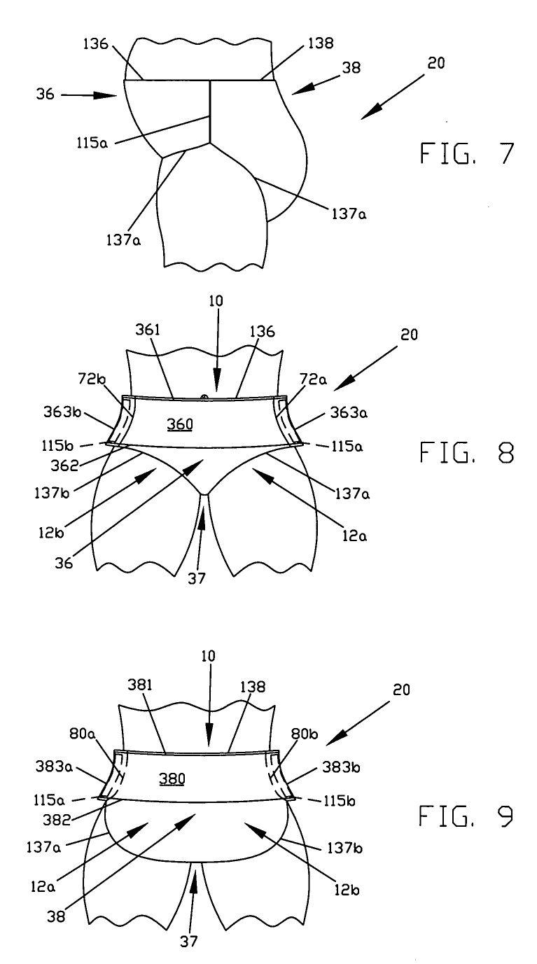

In FIG. 1, FIG. 2, FIG. 3, FIG. 4, FIG. 5, and FIG. 6, the exemplary diaper 20 is shown in its flat uncontracted state prior to being formed into a pant. The finished pant product is shown in FIG. 7, FIG. 8, and FIG. 9.

One end portion of the exemplary diaper 20 is configured as a front waist region 36. The longitudinally opposing end portion is configured as a back waist region 38. An intermediate portion of the diaper 20 extending longitudinally between the front waist region 36 and the back waist region 38 is configured as a crotch region 37.

The basic structure of the diaper 20 includes a chassis 100. The chassis 100 has a laterally extending front waist edge 136 in the front waist region 36 and a longitudinally opposing and laterally extending back waist edge 138 in the back waist region 38. The chassis 100 has a longitudinally extending left side edge 137a and a laterally opposing and longitudinally extending right side edge 137b, both chassis side edges extending longitudinally between the front waist edge 136 and the back waist edge 138. The chassis 100 has an interior surface 102 and an exterior surface 104. The chassis 100 also has a longitudinal axis 42 and a lateral axis 44. The longitudinal axis 42 extends through the midpoint of the front waist edge 136 and through the midpoint of the back waist edge 138. The lateral axis 44 extends through the midpoint of the left side edge 137a and through the midpoint of the right side edge 137b. The exemplary chassis 100 shown in FIG. 1 additionally has longitudinally extending and laterally opposing side flaps 147a and 147b that are described in more detail below.

The basic structure of the diaper 20 also includes an absorbent assembly 200 that is attached to the chassis 100. The absorbent assembly 200 has a laterally extending front edge 236 in the front waist region 36 and a longitudinally opposing and laterally extending back edge 238 in the back waist region 38. The absorbent assembly 200 has a longitudinally extending left side edge 237a and a laterally opposing and longitudinally extending right side edge 237b, both absorbent assembly side edges extending longitudinally between the front edge 236 and the back edge 238. The absorbent assembly 200 has an interior surface 202 and an exterior surface 204. The absorbent assembly 200 may be disposed symmetrically with respect to either or both of the longitudinal axis 42 and the lateral axis 44. Alternatively, the absorbent assembly 200 may be disposed asymmetrically with respect to either or both of the longitudinal axis 42 and the lateral axis 44. For example, the absorbent assembly 200 shown in FIG. 1 is disposed symmetrically with respect to the longitudinal axis 42 and asymmetrically with respect to the lateral axis 44. In particular, the absorbent assembly 200 shown in FIG. 1 is disposed asymmetrically toward the front waist region 36.

The respective front edge 236, back edge 238, left side edge 237a, and right side edge 237b of the absorbent assembly 200 may lie inward of the respective front waist edge 136, back waist edge 138, left side edge 137a, and right side edge 137b of the chassis 100, as in the exemplary diaper 20 shown in FIG. 1. Alternatively, one or more of the edges of the absorbent assembly 200 may coincide with the corresponding edge or edges of the chassis 100.

The basic structure of the diaper 20 also includes at least one abdominal stretch panel that is attached to the chassis 100 in a waist region. When the chassis 100 is stretched in the circumferential direction, the abdominal stretch panel resists by providing a circumferential contractive force around the waist opening of the diaper 20. In FIG. 1, FIG. 2, FIG. 3, FIG. 4, FIG. 5, and FIG. 6, the diaper 20 is shown as having four abdominal stretch panels. In particular, in FIG. 1 and FIG. 3, a front interior abdominal stretch panel 370 is shown attached interiorly to the chassis 100 in the front waist region 36 of the diaper 20. Similarly, in FIG. 1 and FIG. 6, a back interior abdominal stretch panel 390 is shown attached interiorly to the chassis 100 in the back waist region 38 of the diaper 20. In FIG. 2 and FIG. 3, a front exterior abdominal stretch panel 360 is shown attached exteriorly to the chassis 100 in the front waist region 36. Finally, in FIG. 2 and FIG. 6, a back exterior abdominal stretch panel 380 is shown attached exteriorly to the chassis 100 in the back waist region 38 of the diaper 20.

Alternatively, the diaper 20 may have four abdominal stretch panels as described above, or may have only one, two, or three abdominal stretch panels, as desired for providing circumferential contractive forces around the waist of a wearer. For example, in some embodiments, the diaper 20 may have only a front exterior abdominal stretch panel 360, while in other exemplary embodiments, the diaper 20 may have both a front exterior abdominal stretch panel 360 and a back exterior abdominal stretch panel 380. In some exemplary embodiments, the diaper 20 may have only a front interior abdominal stretch panel 370, while in other exemplary embodiments, the diaper 20 may have both a front interior abdominal stretch panel 370 and a back interior abdominal stretch panel 390. In some exemplary embodiments, the diaper 20 may have an exterior abdominal stretch panel and an interior abdominal stretch panel in either the front waist region 36 or the back waist region 38, and only either an exterior abdominal stretch panel or an interior abdominal stretch panel in the opposing waist region.

As shown in FIG. 7, FIG. 8, and FIG. 9, when the diaper 20 is worn on the lower torso of a wearer, the front waist edge 136 and the back waist edge 138 of the chassis 100 encircle the waist of the wearer, while at the same time the chassis side edges 137a and 137b encircle the legs of the wearer. At the same time, the crotch region 37 is generally positioned between the legs of the wearer and the absorbent assembly 200 extends from the front waist region 36 through the crotch region 37 to the back waist region 38.

### Description of the Chassis

The chassis 100 includes a water-impermeable backsheet 26. The backsheet 26 forms an exterior surface that is intended to be placed toward any clothing that is worn over the diaper 20. Many suitable materials for use as the backsheet 26 are well-known, including films of polyethylene and other polyolefins. Multi-layer backsheets, such as a laminate of a film 30 and a nonwoven material 31 or a laminate of multiple nonwoven layers, may also be suitable for use as the backsheet 26. Such a backsheet may be oriented with the nonwoven 31 disposed exteriorly of the film, as shown in FIG. 1, FIG. 2, FIG. 3, FIG. 4, FIG. 5, and FIG. 6, to provide the feel and appearance of a more cloth-like outermost layer than would be provided by using the film 30 as the outermost layer.

As shown in FIG. 1, FIG. 2, FIG. 3, FIG. 4, FIG. 5, and FIG. 6, the exemplary chassis 100 has longitudinally extending and laterally opposing side flaps 147 that are disposed on the interior portion of the diaper 20 that faces inwardly toward the wearer and contacts the wearer. The side flaps 147 may be formed by folding portions of the chassis 100 laterally inward, i.e., toward the longitudinal axis 42, to form both the respective side flaps 147 and the side edges 137 of the chassis 100. Alternatively, the side flaps 147 may be formed by attaching an additional layer or layers to the chassis 100 at or adjacent to each of the respective side edges 137 of the chassis 100.

Each side flap 147 has a proximal edge 157. In the exemplary diaper 20 shown in FIG. 1, the side flaps 147 overlap the absorbent assembly 200, i.e., the proximal edges 157 lie laterally inward of the respective side edges 237 of the absorbent assembly 200. Such an overlapped configuration may be desirable in order to impart a more finished appearance to the diaper 20 than that imparted by a non-overlapped configuration. Alternatively, the side flaps 147 may not overlap the absorbent assembly 200.

Each of the side flaps 147 is attached to the interior surface 102 of the chassis 100 in longitudinally oriented attachment zones 151 in the front waist region 36 and longitudinally oriented attachment zones 152 in the back waist region 38.

In the exemplary chassis 100 shown in FIG. 1, the side flaps 147 are also attached to the interior surface 102 of the chassis 100 in laterally oriented attachment zones 153 adjacent to the front waist edge 136 and in a longitudinally opposing laterally oriented attachment zones 154 adjacent to the back waist edge 138.

In embodiments in which the front edge 236 or the back edge 238 of the absorbent assembly 200 coincides with the respective front waist edge 136 or back waist edge 138 of the chassis 100 and the side flaps 147 overlap the absorbent assembly 200, the side flaps 147 may be attached to the absorbent assembly 200 instead of, or in addition to, being attached to the interior surface 102 of the chassis 100.

Between the attachment zones, the proximal edges 157 of the side flaps 147 remain free, i.e., are not attached to the interior surface 102 of the chassis 100 or to the absorbent assembly 200. Also between the attachment zones, each side flap preferably includes a longitudinally extensible flap elastic gathering member that is attached adjacent to the proximal edge of the side flap by any of many well-known means. Each such flap elastic gathering member may be attached over its entire length or over only a portion of its length. For example, such a flap elastic gathering member may be attached only at or near its longitudinally opposing ends and may be unattached at the middle of its length. Such a flap elastic gathering member may be disposed in the crotch region 37 and may extend into one or both of the front waist region 36 and the back waist region 38. For example, in the exemplary chassis 100 shown in FIG. 1, an elastic strand 167 is attached adjacent to the proximal edge 157 of each of the side flaps 147 and extends into both the front waist region 36 and the back waist region 38.

Each flap elastic gathering member may be enclosed inside a folded hem. For example, in the exemplary chassis 100 shown in FIG. 4 and FIG. 5, each of the elastic strands 167 is enclosed inside a hem 170 formed adjacent to the proximal edge 157 of the respective side flap 147. Alternatively, the flap elastic gathering member may be sandwiched between two layers of the chassis or may be attached on a surface of the chassis 100 and remain exposed.

When stretched, the flap elastic gathering member adjacent to each side flap edge allows the side flap edge to extend to the flat uncontracted length of the chassis, e.g., the length of the chassis 100, as shown in FIG. 1. When allowed to relax, the flap elastic gathering member contracts to pull the front waist region 36 and the back waist region 38 toward each other and thereby bend the diaper 20 into a “U” shape in which the interior of the “U” shape is formed by the portions of the diaper 20 that are intended to be placed toward the body of the wearer. Because each of the proximal edges 157 remains free between the attachment zones 151 and 152, the contractive force of the elastic strand 167 lifts the proximal edge 157 away from the interior surface 102 of the chassis 100. This lifting of the proximal edges 157 when the diaper 20 is in the relaxed condition lifts the side flaps 147 into position to serve as side barriers adjacent to the side edges 237 of the absorbent assembly 200.

At and/or adjacent to each of the side edges 137 of the chassis, the front waist region 36 and the back waist region 38 are attached together to form the finished pant product having a waist opening 10 and two leg openings 12. This attachment may have the form of side seams 115. Such a side seam may be formed where the waist regions are overlapped such that the interior surface of one lies against the exterior surface of the other. In another form, a side seam may be formed where the side edges in the waist regions are abutted. In another form, a side seam may be formed where either the interior surfaces or the exterior surfaces of the waist regions are in face-to-face contact, i.e., in a so-called flanged attachment. Such flanged attachments are shown in FIG. 7, FIG. 8, and FIG. 9.

A portion or the whole of the chassis 100 may be made extensible to a degree greater than the inherent extensibility of the material or materials from which the chassis is made, e.g., the backsheet 26. The additional extensibility may be desirable in order to allow the chassis 100 to conform to the body of a wearer during movement by the wearer. The additional extensibility may also be desirable, for example, in order to allow the user of a diaper 20 including a chassis 100 having a particular size before extension to extend the front waist region 36, the back waist region 38, or both waist regions of the chassis 100 to provide additional body coverage for wearers of differing size, i.e., to tailor the diaper to the individual wearer. Such extension of the waist region or regions may give the diaper a generally hourglass shape, so long as the crotch region 37 is extended to a relatively lesser degree than the waist region or regions, and may impart a tailored appearance to the diaper 20 when it is worn. In addition, the additional extensibility may be desirable in order to minimize the cost of the diaper. For example, an amount of material that would otherwise be sufficient only to make a relatively smaller diaper lacking this extensibility can be used to make a diaper capable of being extended to adequately cover a wearer that is larger than the unextended smaller diaper would fit.

The portion of the chassis 100 underlying an abdominal stretch panel may be made laterally extensible to a maximum extensibility greater than a maximum extensibility of an adjacent portion of the chassis, such that a lateral extension of each of the portions to its maximum extensibility facilitates application of the diaper 20 onto the body of a wearer by enabling the waist opening 10 to be extended to fit over the wearer's hips.

Additional lateral extensibility in the chassis 100 may be provided in a variety of ways. For example, a material or materials from which the chassis 100 is made may be pleated by any of many known methods. Alternatively, all or a portion of the chassis may be made of a formed web material or a formed laminate of web materials like those described in U.S. Pat. No. 5,518,801 issued on 21 May 1996 in the name of Chappell et al. This formed web material includes distinct laterally extending regions in which the original material has been altered by embossing or another method of deformation to create a pattern of generally longitudinally oriented alternating ridges and valleys and also includes laterally extending unaltered regions between the laterally extending altered regions. The formed web material can be extended in a direction perpendicular to the ridges up to the point where the ridges and valleys flatten with substantially less force than is required to extend beyond that point.

The front laterally central portion 117 and the back laterally central portion 118 of the chassis 100 may have a different range of extensibility from the portions of the chassis in the attachment zones 151, 152, 153, and 154 where the side flaps 147a and 147b are attached to the interior surface 102 of the chassis. Additionally or alternatively, the laterally central portions 117 and 118 may be extensible to a greater or lesser degree when subjected to a given level of opposing tensile forces, i.e., may be more easily or less easily extensible, than the portions of the chassis in the attachment zones. For example, if the chassis is made uniformly extensible across its entire width prior to the formation of the side flaps, the double layering in the areas of the attachment zones after the formation of the side flaps may have an effect of decreasing the degree of lateral extensibility of those areas under a given level of opposing tensile forces, such as by the side flaps acting as parallel “springs” that must be extended in order to extend the underlying attached portion of the chassis. As another example, the altered regions in the laterally central portions of the chassis may be deformed to a greater or a lesser degree than the altered regions in the attachment zones to render the laterally central portions more easily or less easily extensible than the respective portions in the attachment zones.

### Description of the Abdominal Stretch Panels

As shown in the figures, each abdominal stretch panel has a circumferentially extending longitudinally distal edge that is disposed adjacent to the respective waist edge of the chassis 100 and a longitudinally opposing circumferentially extending longitudinally proximal edge that is disposed relatively nearer to the lateral axis 44 than the longitudinally distal edge of the same abdominal stretch panel is disposed. Each abdominal stretch panel also has laterally opposing longitudinally extending side edges, an interior surface, and an exterior surface.

In particular, the front interior abdominal stretch panel 370, when present, has a circumferentially extending longitudinally distal edge 371 and a longitudinally opposing circumferentially extending longitudinally proximal edge 372. The front interior abdominal stretch panel 370, when present, also has laterally opposing longitudinally extending side edges 373, an interior surface 374, and an exterior surface 375. Similarly, when present, the back interior abdominal stretch panel 390 has a distal edge 391, a proximal edge 392, side edges 393, an interior surface 394, and an exterior surface 395. Similarly, when present, the front exterior abdominal stretch panel 360 has a distal edge 361, a proximal edge 362, side edges 363, an interior surface 364, and an exterior surface 365. Similarly, when present, the back exterior abdominal stretch panel 380 has a distal edge 381, a proximal edge 382, side edges 383, an interior surface 384, and an exterior surface 385.

Each of the abdominal stretch panels shown in FIG. 1, FIG. 2, FIG. 3, and FIG. 6 has a circumferential extent that is substantially equivalent to the lateral extent of the chassis 100 in the respective waist region. In such embodiments, each abdominal stretch panel may be attached to the chassis 100 where the front waist region 36 and the back waist region 38 are attached together. For example, if a side seam 115 is formed by means of pressure bonding or thermal bonding in any of their forms, including ultrasonic bonding, an abdominal stretch panel may be attached to the chassis in the same side seam, such that the side seam effectively extends through the thicknesses of the chassis 100 and the abdominal stretch panel. An ability to bond all of the layers together in one side seam may have advantages in terms of manufacturing simplicity and cost, because the side seam can be formed in a single bonding process.

Alternatively, any of the abdominal stretch panels may have a circumferential extent that is less than the lateral extent of the chassis 100 in a particular waist region. For example, an interior abdominal stretch panel disposed at and adjacent to the longitudinal axis 42 may have a lateral extent only approximately as great as the distance between the proximal edges 157 of the side flaps 147. As another example, an interior abdominal stretch panel disposed at and adjacent to the longitudinal axis 42 may have a lateral extent greater than the distance between the proximal edges 157 of the side flaps 147 but less than the distance between the side edges 137 of the chassis 100. The portions of such an interior abdominal stretch panel that lie laterally outboard of the proximal edges 157 of the side flaps 147 may be sandwiched between the side flaps 147 and the interior surface 102 of the chassis 100.

The interior surface of each interior abdominal stretch panel contacts the skin of the wearer when the diaper 20 is worn. Therefore, the layer forming the interior surface is preferably formed of a soft material that will not irritate the skin of the wearer. Many suitable materials are known in the art, including rayon and synthetic nonwovens, such as spunbonded or carded polypropylene, polyethylene, or polyester or other olefinic materials.

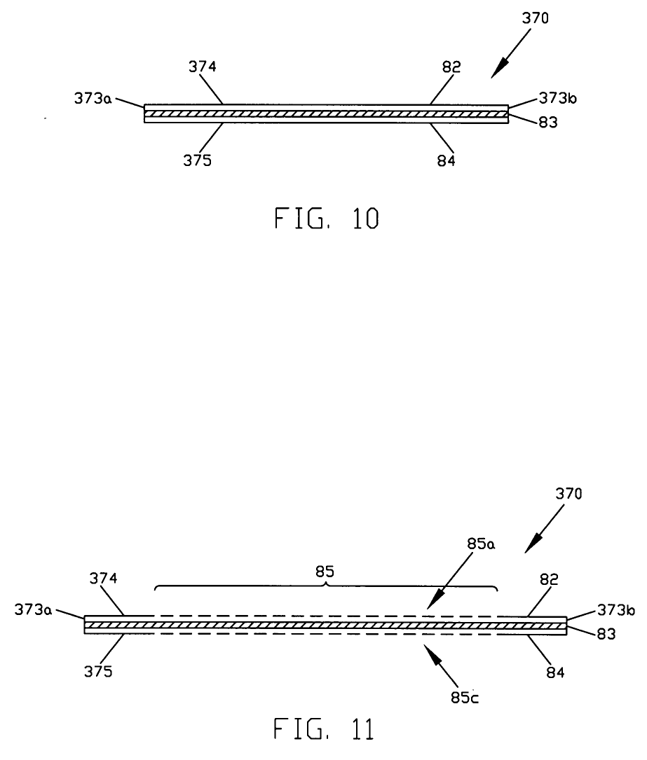

An abdominal stretch panel may have a laminate structure. In particular, an abdominal stretch panel may include an interior layer and an additional layer or layers disposed exteriorly of the interior layer. An elastic version of one of the aforementioned suitable materials, such as a nonwoven exhibiting substantial elastic properties, may be used for any of the layers. For example, a front interior abdominal stretch panel 370 having a laminate structure in shown in FIG. 10. This abdominal stretch panel includes both an interior skin-contacting layer 82 and an elastic layer 83 laminated to the interior layer 82. Suitable materials for the elastic layer 83 are well-known in the art, including natural rubber strands, synthetic rubber strands, elastomeric films, etc. The material chosen for the elastic layer 83 preferably exhibits a force response proportional to its elongation. As shown in FIG. 10, an abdominal stretch panel may also include an exterior cover layer 84 laminated to the elastic layer 83 on its surface opposite the interior layer 82, thereby forming a trilaminate in which the elastic layer 83 is sandwiched between the interior layer 82 and the exterior cover layer 84.

The layers of an abdominal stretch panel may be laminated by any method suitable for the elements being attached together and their constituent materials. For example, the elastic layer 83 may be maintained in a stretched condition while being attached to a relaxed interior layer 82 (and a relaxed exterior cover layer 84 if present) and then allowed to relax. The resultant contraction of the elastic layer 83 may gather the interior layer 82 in such a way as to create rugosities and the laminate thus formed may be extended in the direction of the original stretch up to the original dimension of the interior layer 82 (and the exterior cover layer 84 if present) with only the elastic layer 83 resisting the extension. A similar result may be achieved by, for example, first gathering the interior layer 82 (and the exterior cover layer 84 if present), such as by pleating it, and then attaching the elastic layer 83 in a relaxed condition. The resultant laminate may be extended in a direction perpendicular to the pleat ridges up to the original dimension of the interior layer 82 (and the exterior cover layer 84 if present) with only the elastic layer 83 resisting the extension.

In some exemplary methods, the lamination may be performed with both the elastic layer 83 and the interior layer 82 (and the exterior cover layer 84 if present) relaxed. All or a portion of the resultant laminate abdominal stretch panel may subsequently be “activated” by subjecting it to elongation to create localized ruptures in a portion 85a of the interior layer 82 (and a portion 85c of the exterior cover layer 84 if present). In FIG. 11, a stretched abdominal stretch panel having an activated portion 85 is shown, with the ruptured portion 85a of the interior layer 82 and the ruptured portion 85c of the exterior cover layer 84 shown in dashed lines representing exemplary breaks in and/or separation of the fibers in nonwoven materials. The ruptured portion 85a of the interior layer 82 (and the ruptured portion 85c of the exterior cover layer 84 if present) in the resultant activated portion 85 of the laminate provides little or no resistance to extension in the direction of the original elongation. For example, when a nonwoven is used for the interior layer 82 (and the exterior cover layer 84 if present), the ruptured portion(s) typically include(s) breaks in and/or separation of the fibers that render the ruptured portion(s) substantially incapable of transmitting tensile forces in the plane of the nonwoven. Some suitable activation methods are known in the art as “ring-rolling” processes.

A combination of lamination methods may be used, if desired, so long as they are suitable for the elements being attached together and their constituent materials.

An abdominal stretch panel may have uniform extension characteristics throughout its area or may have different extension characteristics in different portions. For example, a portion of an abdominal stretch panel may be made laterally extensible to a maximum extensibility greater than a maximum extensibility of an adjacent portion, such that a desired fit on a wearer can be achieved. The difference in extensibility may be provided by varying the lamination process, for example by varying the localized elongation in an activation process.

Some users may desire to tear open the diaper 20 in order to remove it from the body of the wearer. In order to provide guidance regarding where the product may be torn open, a visible tear locator line may be printed on an abdominal stretch panel and the adjacent portion of the chassis 100. For example, a pair of laterally opposing tear locator lines 72a, 72b may be printed exteriorly on a front exterior abdominal stretch panel 360 as shown in FIG. 8. Tearing an exterior abdominal stretch panel and the underlying portion of the chassis 100 (and the underlying interior abdominal stretch panel, if present) at both of two laterally opposing tear locator lines will release the diaper 20 from the waist and both legs of the wearer.

Such a tear locator line 72 may be printed on any abdominal stretch panel(s). For some users, locating the tear locator lines 72 on a front exterior abdominal stretch panel may be preferable, because they may prefer to lay their infants down on their backs while removing the diaper, and tear locator lines 72 disposed on a front abdominal stretch panel are likely to be more easily visible than tear locator lines 72 disposed on a back abdominal stretch panel.

In order to minimize the level of tensile force required to tear open the diaper 20 for removal from the wearer, frangible tear lines may be provided. Such a frangible tear line may be formed in a layer or a laminate of layers by perforation, by the formation of a brittle area or areas at which the material will preferentially fracture when stressed, by the formation of a weaker area or areas at which the material will preferentially tear when stressed, by the formation of a friable area or areas at which the material will preferentially crumble when stressed and/or bent, or by any other method of providing frangibility that is suitable for the materials involved.

For example, a pair of laterally opposing frangible tear lines 80a, 80b may be formed in a back exterior abdominal stretch panel 380 as well as the portion of the chassis 100 overlain by the back abdominal stretch panel, as shown in FIG. 9. In this embodiment, the frangible tear lines 80 intersect the distal edge 381 of the back exterior abdominal stretch panel 380 laterally inboard of its side edges 383 and intersect its proximal edge 382 at the intersection of the proximal edge 382 and the side edge 137 of the chassis 100 at the margin of each leg opening. The frangible tear lines 80 may be disposed elsewhere, but the depicted configuration may be preferred in some embodiments. For example, if the frangible tear lines 80 continued generally linearly toward the lateral axis 44, continuous portions of the diaper 20 would remain in place around the legs of the wearer. When the frangible tear lines 80 are shaped to intersect the margins of the respective leg openings as described above, tearing the diaper 20 at both of the laterally opposing frangible tear lines 80 will release the diaper 20 from the waist and both legs of the wearer.

Such frangible tear lines 80 may be disposed in any abdominal stretch panel(s). For some users, locating the frangible tear lines 80 in a front abdominal stretch panel may be preferable, because they may prefer to lay their infants down on their backs while removing the diapers, and frangible tear lines 80 in a front abdominal stretch panel are likely to be more easily visible and more easily usable than frangible tear lines 80 disposed in a back abdominal stretch panel. If both an exterior abdominal stretch panel and an interior abdominal stretch panel are present in a waist region, the frangible tear lines may be formed in both of the abdominal stretch panels as well as the portion of the chassis 100 sandwiched between them.

In some embodiments, both frangible tear lines and tear locator lines may be provided. For example, tear locator lines may be printed at or adjacent to frangible tear lines. In such an embodiment, the presence of the visible tear locator lines may prove helpful to the user in locating the frangible tear lines for use in removal of the diaper 20.

The abdominal stretch panels attached to the chassis as described herein are desirable from the standpoints of comfort and appearance. For example, unlike typical stretch waistbands, each abdominal stretch panel covers some portion of a waist region of the diaper 20, rather than being hidden between layers of the chassis 100. Therefore, if the abdominal stretch panel is formed from soft and attractive materials, such as one of the aforementioned nonwovens, the exposed abdominal stretch panel can provide a finished appearance resembling that of cloth underwear and thereby convey an impression of softness and comfort to the user.

### Description of the Absorbent Assembly

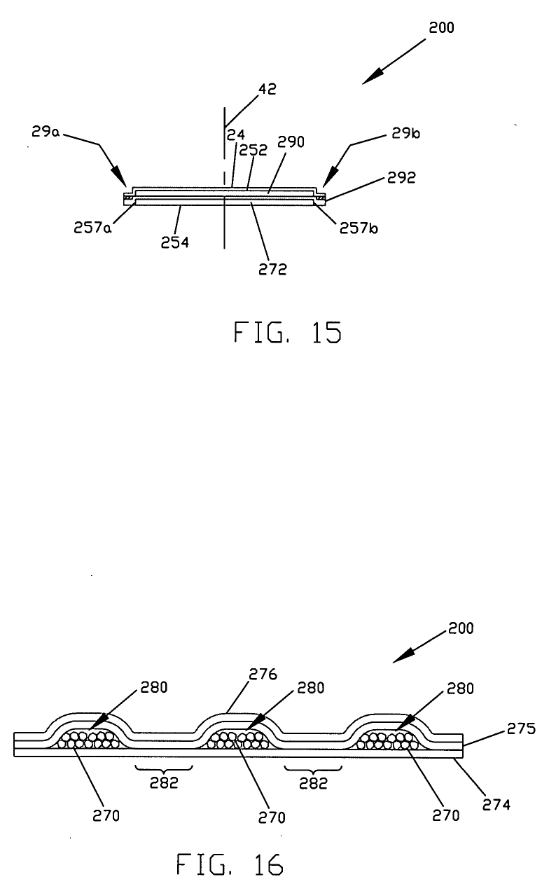

As shown in FIG. 14, FIG. 15, and FIG. 16, the absorbent assembly 200 includes an absorbent core 250 that serves to absorb and retain liquid bodily waste materials. The absorbent core 250 has a laterally extending front edge 256 and a longitudinally opposing and laterally extending back edge 258. The absorbent core 250 also has a longitudinally extending left side edge 257a and a laterally opposing and longitudinally extending right side edge 257b, both absorbent core side edges extending longitudinally between the front edge 256 and the back edge 258. The absorbent core 250 also has an interior surface 252 and an exterior surface 254.

The absorbent assembly 200 may be attached to the interior surface 102 of the chassis 100 over any part or the whole of the area of the absorbent assembly 200. Preferably, the absorbent assembly 200 is attached on its exterior surface 204 to the chassis 100 in a cruciform attachment pattern, i.e., in an attachment pattern that forms or is arranged in a cross or “+” shape. The cruciform attachment pattern may be contiguous, i.e., all of its portions may be touching or connected throughout the pattern in an unbroken sequence. Alternatively, the cruciform attachment pattern may include detached portions and thereby lack contiguity but still be arranged such that the shape of the overall pattern is a cruciform. For example, a discontiguous cruciform attachment pattern may include a longitudinally extending portion disposed along the longitudinal axis and separate left and right laterally distal portions disposed along or adjacent to the lateral axis and thereby form a cruciform as the shape of the overall pattern.

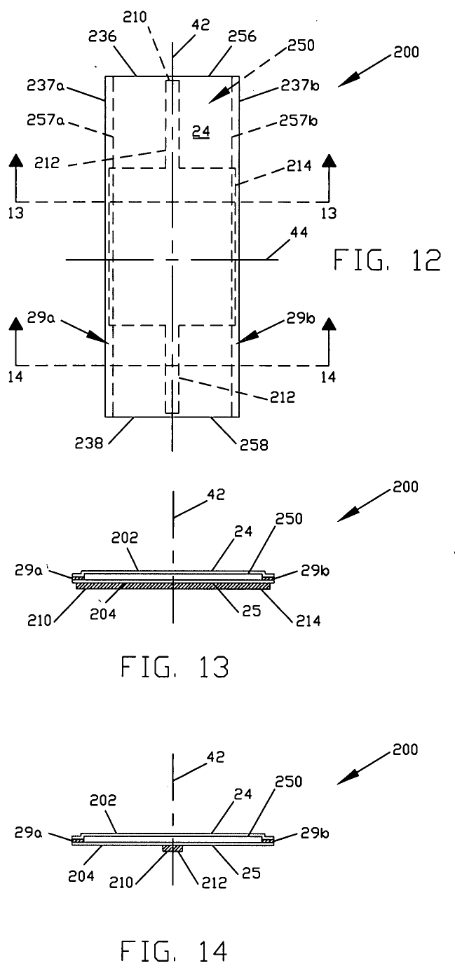

An exemplary contiguous cruciform attachment pattern 210 is shown in FIG. 2, FIG. 4, FIG. 5, FIG. 12, FIG. 13, and FIG. 14. The portions of the chassis 100 that lie outside such a cruciform attachment pattern are not restrained by attachment to the absorbent assembly 200 and therefore remain extensible. In particular, a relatively narrow longitudinally extending portion 212 of a cruciform attachment pattern 210 like that shown in these figures leaves the majority of the width of the chassis 100 in the front waist region 36 and in the back waist region 38 freely extensible and thereby allows extension of the chassis 100 in the lateral direction in these regions. A relatively wide laterally extending portion 214 of a cruciform attachment pattern 210 like that shown in these figures prevents the portion of the chassis 100 in the crotch region 37 to which the absorbent assembly 200 is attached from shifting relative to the absorbent assembly 200 in that region. A relatively wide laterally extending portion 214 of a cruciform attachment pattern 210 may also contribute to the effectiveness and positioning of the side flaps 147 when the elastic strands 167 lift the proximal edges 157 into contact with the body of the wearer. Because the relatively wide laterally extending portion 214 of the cruciform attachment pattern 210 restrains the chassis 100 over a relatively wide portion of the width of the crotch region 37, the side flaps 147 are more likely to remain properly positioned while being lifted by the elastic strands 167.

Within the extent of the cruciform attachment pattern 210, the absorbent assembly 200 may be attached to the chassis 100 continuously or intermittently. For example, a film of an adhesive material may be applied continuously over the entire area of the cruciform attachment pattern and then used to continuously attach the absorbent assembly to the chassis. As an alternative example, an adhesive material may be applied discontinuously at and inside the boundaries of the cruciform attachment pattern, such as in the form of dots, stripes, beads, spirals, etc., and then used to attach the absorbent assembly to the chassis.

The cruciform attachment pattern 210 may be disposed symmetrically or asymmetrically with respect to either or both of the longitudinal axis 42 and the lateral axis 44 of the chassis 100. In addition, the cruciform attachment pattern 210 may be disposed symmetrically or asymmetrically with respect to either or both of the side edges 237 and the front edge 236 and the back edge 238 of the absorbent assembly 200.

Suitable configurations of cruciform attachment patterns are disclosed in U.S. Pat. No. 6,962,578 issued on 8 Nov. 2005.

The absorbent core 250 may be disposed between a lower covering sheet that is disposed on the exterior face of the absorbent core 250 and an upper covering sheet that is disposed on the interior face of the absorbent core 250. Such an upper covering sheet and lower covering sheet may be attached together to contain the absorbent core 250 between them and thereby form the absorbent assembly 200. For example, in the exemplary absorbent assembly 200 shown in FIG. 12, FIG. 13, and FIG. 14, an upper covering sheet 24 and a lower covering sheet 25 are attached together at or adjacent to the side edges 237 of the absorbent assembly 200 in longitudinally extending attachment zones 29. Alternatively, the upper covering sheet 24 and the lower covering sheet 25 may be attached together in places other than the side edges 237, e.g., at or adjacent to the end edges 236 and 238 of the absorbent assembly 200, or at or adjacent to both the end edges 236 and 238 and the side edges 237. Both the upper covering sheet and the lower covering sheet are water vapor-permeable, i.e., breathable.

The upper covering sheet 24 is water-permeable and allows liquid waste to pass through to the absorbent core 250, where the liquid waste is absorbed. The lower covering sheet 25 may be water-impermeable. However, the lower covering sheet 25 is preferably water-permeable.

In the exemplary absorbent assembly 200 shown in FIG. 12, FIG. 13, and FIG. 14, the upper covering sheet 24 and the lower covering sheet 25 are of the same size, i.e., both the upper covering sheet 24 and the lower covering sheet 25 extend to the front edge 236 and back edge 238, as well as to the side edges 237 of the absorbent assembly 200. Alternatively, the upper covering sheet 24 and the lower covering sheet 25 may differ in size.

As another example, the lower covering sheet 25 may be larger than the upper covering sheet 24 and may be wrapped over the side edges 257 of the absorbent core 250 onto the interior surface of the absorbent core 250, where the upper covering sheet 24 and the lower covering sheet 25 may be attached together. Alternatively, in place of a separate upper covering sheet 24 and a separate lower covering sheet 25, a single covering sheet may be wrapped around the absorbent core 250 and attached to itself to contain the absorbent core 250.

The absorbent core 250 includes a storage component 272 that serves to absorb and retain liquid bodily waste materials. Suitable known materials for the absorbent core storage component include cellulose fibers in the form of comminuted wood pulp, commonly known as “airfelt”, natural or synthetic fibrous materials, and superabsorbent polymers, used either singly or in mixtures and commonly formed into layers or sheets, etc.

The absorbent core may include an acquisition component in addition to one or more storage components. The absorbent core acquisition component serves to acquire deposited liquid bodily waste material and transfer it to the absorbent core storage component. Any porous absorbent material which will imbibe and partition liquid bodily waste material to the storage component or components may be used to form the acquisition component. Examples of such acquisition materials are more fully described in U.S. Pat. No. 4,950,264 issued to Osborn on Aug. 21, 1990.

Such an absorbent core acquisition component 290 is shown overlying the absorbent core storage component 272 in FIG. 15. A separation sheet 292 of, e.g., a tissue or a nonwoven material, may be disposed between the absorbent core storage component 272 and the absorbent core acquisition component 290 to help ensure that none of the gel formed by a superabsorbent polymer that may be included in the absorbent core storage component reaches the skin of the wearer.

In some exemplary embodiments, an absorbent core storage component may include the discrete form of an absorbent material that is immobilized in pockets formed by a layer of a thermoplastic material, such as a hot melt adhesive material, that intermittently contacts and adheres to a substrate sheet, while diverging away from the substrate sheet at the pockets. Absorbent core components having such structures and being suitable for the storage of liquid bodily wastes are described in U.S. Patent Application Publication No. 2004/0162536 dated 19 Aug. 2004 and U.S. Patent Application Publication No. 2004/0167486 dated 26 Aug. 2004. An exemplary absorbent core storage component 272 having such a structure is shown in FIG. 16. In this absorbent core storage component 272, particles 270 of a superabsorbent polymer are contained inside pockets 280 formed by a layer 275 of a thermoplastic material. The layer 275 of the thermoplastic material intermittently contacts and adheres to a substrate sheet 274 at the areas of attachment 282. Between the areas of attachment 282, the layer 275 diverges away from the substrate sheet 274 to form the pockets 280. The layer 275 may have the form of a sheet of fibers of the thermoplastic material through which the liquid waste may pass to the particles to be absorbed by the particles 270 of the superabsorbent polymer.

In FIG. 16, a separate thermoplastic layer covering sheet 276 is shown overlying the layer 275 of the thermoplastic material. Alternatively, the separate thermoplastic layer covering sheet 276 may be omitted.

### Statements of Incorporation by Reference and Intended Scope of Claims

The disclosures of all patents, patent applications and any patents which issue thereon, as well as any corresponding published foreign patent applications, and all publications listed and/or referenced in this description, are hereby incorporated herein by reference. It is expressly not admitted that any of the documents or any combination of the documents incorporated herein by reference teaches or discloses the present invention. To the extent that any meaning or definition of a term in this document conflicts with any meaning or definition of the same term in a document incorporated by reference, the meaning or definition assigned to that term in this document shall govern.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A disposable diaper comprising:
an absorbent assembly comprising an absorbent core;
a chassis having a longitudinal axis, a lateral axis, a front waist region having a front waist edge, a back waist region having a back waist edge, a crotch region between the waist regions, laterally opposing side edges extending between the front waist edge and the back waist edge, an exterior surface, and an interior surface to which the absorbent assembly is attached, the chassis comprising a water-impermeable backsheet and laterally opposing side flaps attached to the interior surface adjacent to their longitudinally distal ends, each of the side flaps having a longitudinally extending elastic gathering member attached adjacent to its proximal edge, the front waist region and the back waist region being attached together at or adjacent to the respective side edges and thereby forming a waist opening and two laterally opposing leg openings; and
at least one abdominal stretch panel attached to the interior surface or the exterior surface in the front waist region or the back waist region and having a circumferentially extending longitudinally distal edge disposed adjacent to the respective waist edge and a longitudinally opposing circumferentially extending longitudinally proximal edge, the abdominal stretch panel providing a circumferential contractive force around the waist opening when the chassis is stretched circumferentially.

2. The disposable diaper of claim 1 wherein the abdominal stretch panel includes an elastic material.

3. The disposable diaper of claim 1 wherein the abdominal stretch panel includes a skin-contacting layer and an elastic layer laminated to the skin-contacting layer.

4. The disposable diaper of claim 1 wherein the abdominal stretch panel comprises a trilaminate including an interior layer, an exterior cover layer, and an elastic layer sandwiched between the interior layer and the exterior cover layer.

5. The disposable diaper of claim 4 wherein the trilaminate includes an activated portion in which a ruptured portion of the interior layer and a ruptured portion of the exterior cover layer provide substantially no resistance to circumferential elongation and the elastic layer provides a circumferential contractive force.

6. The disposable diaper of claim 1 wherein the abdominal stretch panel includes laterally opposing tear locator lines printed exteriorly on the abdominal stretch panel and extending from the distal edge of the abdominal stretch panel to the proximal edge of the abdominal stretch panel.

7. The disposable diaper of claim 6 wherein the abdominal stretch panel is attached onto the exterior surface of the chassis in the front waist region.

8. The disposable diaper of claim 1 further comprising laterally opposing frangible tear lines formed in the abdominal stretch panel and the chassis and extending from the distal edge of the abdominal stretch panel to the proximal edge of the abdominal stretch panel such that tearing along the frangible tear lines releases the diaper from the waist and legs of a wearer's body.

9. The disposable diaper of claim 8 wherein the abdominal stretch panel is attached onto the exterior surface of the chassis in the front waist region.

10. The disposable diaper of claim 1 wherein the absorbent assembly is attached to the chassis in a cruciform attachment pattern.

11. The disposable diaper of claim 10 wherein at least a portion of the chassis lying outside the cruciform pattern is laterally extensible.

12. The disposable diaper of claim 1 wherein at least a portion of the chassis in the front waist region or the back waist region is laterally extensible.

13. The disposable diaper of claim 1 wherein the abdominal stretch panel comprises an extensible nonwoven.

14. The disposable diaper of claim 13 wherein the extensible nonwoven is elastically extensible.

15. The disposable diaper of claim 1 wherein the abdominal stretch panel comprises a nonwoven layer and an elastic layer laminated to the nonwoven layer.

16. The disposable diaper of claim 1 wherein the abdominal stretch panel comprises a nonwoven.

17. The disposable diaper of claim 2 further comprising laterally opposing frangible tear lines formed in the abdominal stretch panel and the chassis and extending from the distal edge of the abdominal stretch panel to the proximal edge of the abdominal stretch panel such that tearing along the frangible tear lines releases the diaper from the waist and legs of a wearer's body.

18. A disposable diaper comprising:
an absorbent assembly comprising an absorbent core;
a chassis having a longitudinal axis, a lateral axis, a front waist region having a front waist edge, a back waist region having a back waist edge, a crotch region between the waist regions, laterally opposing side edges extending between the front waist edge and the back waist edge, an exterior surface, and an interior surface to which the absorbent assembly is attached, the chassis comprising a water-impermeable backsheet and laterally opposing side flaps attached to the interior surface adjacent to their longitudinally distal ends, each of the side flaps having a longitudinally extending elastic gathering member attached adjacent to its proximal edge, the front waist region and the back waist region being attached together at or adjacent to the respective side edges and thereby forming a waist opening and two laterally opposing leg openings; and
a front abdominal stretch panel attached to the exterior surface in the front waist region and a back abdominal stretch panel attached to the exterior surface in the back waist region, each of the abdominal stretch panels having a circumferentially extending longitudinally distal edge disposed adjacent to the respective waist edge and a longitudinally opposing circumferentially extending longitudinally proximal edge, and each of the abdominal stretch panels providing a circumferential contractive force around the waist opening when the chassis is stretched circumferentially.

19. The disposable diaper of claim 18 wherein at least one of the abdominal stretch panels extends laterally between the side edges of the chassis.

20. A disposable diaper comprising:
an absorbent assembly comprising an absorbent core;
a chassis having a longitudinal axis, a lateral axis, a front waist region having a front waist edge, a back waist region having a back waist edge, a crotch region between the waist regions, laterally opposing side edges extending between the front waist edge and the back waist edge, an exterior surface, and an interior surface to which the absorbent assembly is attached, the chassis comprising a water-impermeable backsheet and laterally opposing side flaps attached to the interior surface adjacent to their longitudinally distal ends, each of the side flaps having a longitudinally extending elastic gathering member attached adjacent to its proximal edge, the front waist region and the back waist region being attached together at or adjacent to the respective side edges and thereby forming a waist opening and two laterally opposing leg openings;
a front interior abdominal stretch panel attached to the interior surface in the front waist region, a front exterior abdominal stretch panel attached to the exterior surface in the front waist region, a back interior abdominal stretch panel attached to the interior surface in the back waist region, and a back exterior abdominal stretch panel attached to the exterior surface in the back waist region, each of the abdominal stretch panels having a circumferentially extending longitudinally distal edge disposed adjacent to the respective waist edge and a longitudinally opposing circumferentially extending longitudinally proximal edge, and each of the abdominal stretch panels providing a circumferential contractive force around the waist opening when the chassis is stretched circumferentially.
